(19) United States
(12) Patent Application Publication
(10) Pub. No.: US 2005/0136458 A1
(43) Pub. Date: Jun. 23, 2005

(54) Method for nucleic acid preparation

(75) Inventors: Roderic M.K. Dale, Wilsonville, OR (US); Steven L. Gatton, Lake Oswego, OR (US)

Correspondence Address: WILSON SONSINI GOODRICH & ROSATI, 650 PAGE MILL ROAD, PALO ALTO, CA, 943041050, US

(73) Assignees: Oligos, Etc, Inc.; Oligo Therapeutics Inc.

(21) Appl. No.: 10/984,030

(22) Filed: Sep. 14, 2004

Related U.S. Application Data
(63) Continuation of application No. 09/223,957, filed on Dec. 31, 1998, now abandoned.

(51) Int. Cl. C12Q 1/68; C07H 21/04

(52) U.S. Cl. 435/6; 536/25.4

(57) ABSTRACT

Concentration of oligonucleotides in salt solutions is accomplished by loading a solution of the oligonucleotide dissolved in aqueous sodium chloride or other salt solution onto a reverse-phase poly(styrene-divinylbene) liquid chromatography (LC) column. The column bearing oligonucleotide is then washed with water to low conductivity and eluted with an organic eluent such as ethanol, thus effecting a combination desalting concentration procedure in one step, thus this procedure has utility in the desalting and concentration of oligonucleotides that have been purified and/or treated by anion exchange chromatography. In situ cationic exchange of the associated cation of the oligonucleotide can also be incorporated into the procedure of the new invention.

### FIELD OF THE INVENTION

The present invention relates to the processes and reagents for concentrating and desalting nucleic acids from aqueous salt solutions.

### BACKGROUND TO THE INVENTION

Over the past several years the use of oligonucleotides in molecular biology and related disciplines has become a rapidly expanding technique. The manufacture of such oligonucleotides ranges in amounts from less than a milligram for research and testing to the kilogram quantities required for oligonucleotide-based pharmaceuticals.

One characteristic of oligonucleotide synthesis is the formation of truncated, less-than-fill-length chains that result from the synthesis process. These “failure” sequences present the most formidable challenge for purification of the crude oligonucleotides. While there are several methods for attempting to remove these “short-mers”, there are drawbacks to each. In either strong anion exchange (SAX) or weak anion exchange (WAX) chromatography purification, longer oligonucleotides require higher concentrations of aqueous salts to elute from the column, with the resulting benefit that shorter failure sequences elute before the desired full-length oligonucleotide. See, e.g., Liautard J. Chromatogr. 476:439-43 (1989), Dion et. al, J. Chromatogr. 535:127-45 (1990); Gerstner et al., Nucleic Acids Res. 23:2292-99 (1995); Ausserer and Biros, Biotechniques 19:136-9 (1995). While this technique can be quite successful at separating out short-mers, the fill-length oligonucleotides must be desalted and concentrated from the elute before use in most techniques.

A number of methods exist for concentrating and desalting size restricted purified oligonucleotides, including reverse phase capture, precipitation, size exclusion chromatography, diafiltration, and electrodialysis.

The technique of reverse phase capture for desalting oligonucleotides uses selective absorption of an oligonucleotide from an aqueous salt solution as that solution passes through a reverse-phase liquid chromatography column. Current practice of this technique is limited by the relatively weak absorption of the oligonucleotide by any reverse-phase solid phase. Because of this weak absorption, the oligonucleotide begins to leach off the column as the salt concentration begins to drop below that of the initial simple solution. As a result, the eluted sample must contain significant amounts of salt, which must be removed by, further desalting. One well-known technique to alleviate this problem is to replace the salt from the anion exchange with a volatile salt such as ammonium acetate. Washing the column bearing oligonucleotide with a solution of that volatile salt is done in a manner to maintain polarity of the loading solution. The elution of the oligonucleotide is then carried out with a buffer system with sufficient volatile salt in the phases to maintain the absorption until the elution point is reached. Excess volatile salt is then removed during lyophilization. The principal drawback of this variation is that useful cations that are not available as volatile salts (i.e., sodium, potassium) must be introduced by cation exchange in a separate operation.

Precipitation of an oligonucleotide, which necessarily follows most available purification methods, involves adding ethanol or similar solvent to a salt solution of the oligonucleotide, followed by centrifugation and washing the precipitate. The technique does not work well for smaller oligonucleotides (<10-mer) and is difficult to scale up from benchtop scale because of the expensive centrifugation equipment required for industrial production. Removal of residual salts and solvents also presents a problem, particularly in large scale operations.

Size exclusion chromatography (SEC) requires pre-concentration of the oligonucleotide solution as a separate step prior to the desalting. It results in only limited desalting of smaller oligonucleotides and regardless of size leads to dilution of the oligonucleotide. In addition, many SEC column packing materials leach contaminating material into the oligonucleotide.

Diafiltration is based on the size differential between small salt ions and larger molecules, such as oligonucleotides. Diafiltration is in effect a filtering away of the salt ions through a microporous membrane, assisted by low pressure. While this is a well-utilized technique of desalting proteins, concentration of the nucleic acids is only moderate at best, leaving large quantities of solution. In addition, an oligonucleotide molecule presents a relatively small dimension and, if oriented properly, it can pass through the membrane almost as easily as the smaller-mass ions. This can result in unacceptable loss of product across the diafiltration membrane. Diafiltration is also very slow, and can take many hours to achieve acceptable salt reduction. The membranes are prone to clogging and can be difficult to sanitize.

Electrodialysis is similar in concept to diafiltration, except that the driving force of the filtration is electrostatic interactions rather than pressure. Limitations of diafiltration due to molecular dimensions limit this technique as well.

There remains a need in the art for a more efficient and effective way of concentrating and desalting oligonucleotides following size selection purification. In particular, there is a need for a fast, reproducible method that is effective for both small scale and large scale production of oligonucleotides.

### SUMMARY OF THE INVENTION

The present invention provides a method of concentrating and desalting nucleic acids (e.g. oligonucleotides). The method comprises purifying the nucleic acid from a sample by (1) running the sample over a binding medium comprising a binding material, e.g., poly(styrene-divinylbenzene), (2) allowing the nucleic acid to bind to the medium, and (3) eluting the nucleic acid in a desired volume of an aqueous organic solvent.

In a preferred embodiment of the invention, the concentration and desalting process also involves rinsing the binding medium following binding of the nucleic acid with an unbuffered aqueous solution, preferably water, before eluting the nucleic acid with the organic solvent. This rinsing step functions to remove any unbound impurities, e.g., salts used in previous processing and/or purification steps, allowing the oligonucleotide to remain attached while the salt concentration in the binding medium is lowered. Preferably, the rinsing with the unbuffered aqueous solution (e.g., water) results in a the effluent having a conductivity of at or below 100 microSiemens/cm following rinsing but prior to elution of the oligonucleotide.

An advantage of the method of the invention is that it functions well with nucleic acids comprised of naturally occurring bases and/or altered synthetic bases.

Another advantage of the method of the invention is that it works well with nucleic acids having various modifiers such as biotin, fluoreicen and related dyes, spacers, thiol modifiers, amino modifiers, carboxylate modifiers, or any combination of these.

A feature of this method is that the techniques can be applied to almost any scale of operation.

### DETAILED DESCRIPTION OF THE PREFERRED EMBODIMENTS

It is to be understood that this invention is not limited to the particular methodology, protocols, and reagents described, as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “bacteria” may include a plurality of bacterial species and “an oligonucleotide” may encompass a plurality of oligonucleotides and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned are incorporated herein by reference for the purpose of describing and disclosing, for example, the methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### Definitions

The terms “nucleic acid” and “nucleic acid molecule” as used interchangeably herein, refer to a molecule comprised of nucleotides, i.e., ribonucleotides, deoxyribonucleotides, or both. The term includes monomers and polymers of ribonucleotides and deoxyribonucleotides, with the ribonucleotide and/or deoxyribonucleotides being connected together, in the case of the polymers, via 5′ to 3′ linkages. However, linkages may include any of the linkages known in the nucleic acid synthesis art including, for example, nucleic acids comprising 5′ to 2′ linkages. The nucleotides used in the nucleic acid molecule may be naturally occurring or may be synthetically produced analogues that are capable of forming base-pair relationships with naturally occurring base pairs. Examples of non-naturally occurring bases that are capable of forming base-pairing relationships include, but are not limited to, aza and deaza pyrimidine analogues, aza and deaza purine analogues, and other heterocyclic base analogues, wherein one or more of the carbon and nitrogen atoms of the purine and pyrimidine rings have been substituted by heteroatoms, e.g., oxygen, sulfur, selenium, phosphorus, and the like.

The term “oligonucleotide” as used herein refers to a nucleic acid molecule comprising from about 1 to about 100 nucleotides, more preferably from 1 to 80 nucleotides, and even more preferably from about 4 to about 35 nucleotides.

The term “monomer” as used herein refers to a nucleic acid molecule and derivatives thereof comprised of a single nucleotide.

The terms “modified oligonucleotide”, “modified monomer”, and “modified nucleic acid molecule” as used herein refer to nucleic acids with one or more chemical modifications at the molecular level of the natural molecular structures of all or any of the nucleic acid bases, sugar moieties, intemucleoside phosphate linkages, as well as molecules having added substituents, such as diamines, cholesteryl or other lipophilic groups, or a combination of modifications at these sites. The intemucleoside phosphate linkages can be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate and/or sulfone internucleotide linkages, or 3′-3′, 2′-5′, or 5′-5′ linkages, and combinations of such similar linkages (to produce mixed backbone modified oligonucleotides). The modifications can be internal (single or repeated) or at the end(s) of the oligonucleotide molecule and can include additions to the molecule of the internucleotide phosphate linkages, such as cholesteryl, diamine compounds with varying numbers of carbon residues between amino groups and terminal ribose, deoxyribose and phosphate modifications which cleave or cross-link to the opposite chains or to associated enzymes or other proteins. Electrophilic groups such as ribose-dialdehyde could covalently link with an epsilon amino group of the lysyl-residue of such a protein. A nucleophilic group such as n-ethylmaleimide tethered to an oligomer could covalently attach to the 5′ end of an MRNA or to another electrophilic site. The term modified oligonucleotides also includes oligonucleotides comprising modifications to the sugar moieties such as 2′-substituted ribonucleotides, or deoxyribonucleotide monomers, any of which are connected together via 5′ to 3′ linkages. Modified oligonucleotides may also be comprised of PNA or morpholino modified backbones where target specificity of the sequence is maintained.

The term “nucleic acid backbone” as used herein refers to the structure of the chemical moiety linking nucleotides in a molecule. This may include structures formed from any and all means of chemically linking nucleotides. A modified backbone as used herein includes modifications to the chemical linkage between nucleotides, as well as other modifications that may be used to enhance stability and affinity, such as modifications to the sugar structure. For example an α-anomer of deoxyribose may be used, where the base is inverted with respect to the natural β-anomer. In a preferred embodiment, the 2′-OH of the sugar group may be altered to 2′-O-alkyl or 2′-O-alkyl-n(O-alkyl), which provides resistance to degradation without comprising affinity.

The term “acidification” and “protonation/acidification” as used interchangeably herein, refers to the process by which protons (or positive hydrogen ions) are added to proton acceptor sites on a nucleic acid. The proton acceptor sites include the amine groups on the base structures of the nucleic acid and the phosphate of the phosphodiester linkages. As the pH is decreased, the number of these acceptor sites which are protonated increases, resulting in a more highly protonated/acidified nucleic acid.

The term “protonated/acidified nucleic acid” refers to a nucleic acid that, when dissolved in water at a concentration of approximately 16 A₂₆₀ per ml, has a pH lower than physiological pH, i.e., lower than approximately pH 7. Modified nucleic acids, nuclease-resistant nucleic acids, and antisense nucleic acids are meant to be encompassed by this definition. Generally, nucleic acids are protonated/acidified by adding protons to the reactive sites on a nucleic acid, although other modifications that will decrease the pH of the nucleic acid can also be used and are intended to be encompassed by this term.

The term “end-blocked” as used herein refers to a nucleic acid with a chemical modification at the molecular level that prevents the degradation of selected nucleotides, e.g., by nuclease action. This chemical modification is positioned such that it protects the integral portion of the nucleic acid, for example the coding region of an antisense oligonucleotide. An end block may be a 3′ end block or a 5′ end block. For example, a 3′ end block may be at the 3′-most position of the molecule, or it may be internal to the 3′ ends, provided it is 3′ to the integral sequences of the nucleic acid.

The term “effluent” as used herein refers to a liquid sample obtained following exposure to a binding material with adsorbed nucleic acid. For example, an effluent may be an aqueous solvent exposed to a liquid chromatography column containing adsorbed oligonucleotide. The effluent may be collected following elution of the nucleic acid from the binding material, in which case the effluent will contain the eluted nucleic acid in solution. Alternatively, a “rinse effluent” may contain salts removed from the binding material prior to the elution of the nucleic acid from the binding material, but negligible amounts of the bound nucleic acid.

### The Invention in General

The present invention provides a protocol with methods and reagents which when used in the concentrating and desalting procedure will contribute to the overall efficiency of size selection purification methods, such as anion exchange chromatography. In a preferred embodiment liquid chromatography (LC) columns packed with materials that strongly adhere to nucleic acids, such as poly(styrene-divinylbenzene), can be used to selectively absorb nucleic acids, and particularly oligonucleotides, from aqueous salt solutions. This absorption on this type of solid support is strong enough to allow the use of unbuffered water to wash the salt from the column. The oligonucleotide can be eluted from the column using a compatible aqueous unbuffered organic solvent, either isocratically or as a gradient, resulting in the oligonucleotide being concentrated in a desalted solution. The desalted solution can then be easily lyophilized to yield the pure, desalted oligonucleotide in a dried form.

The method of the invention can be applied to almost any scale of operation. With slight modifications dictated by the requirements of safe operation of the process equipment, the procedure of the new invention can be used for submilligram to kilogram scale. Chromatographic equipment ranging from conventional HPLCs, a Pharmacia BioPilot, and Amicon K40 sanitary LC's can be used for this procedure. As such, scale-up from bench through production is essentially limited only by the capacity of the equipment available.

The present invention is not limited to synthetic DNA phosphodiester oligonucleotides, and can be used successfully with oligonucleotides with modified backbones such as phosphorothioates, RNA, 2′-O-methyl RNA and other 2′-O-alkyl RNA, methylphosphonates, p-ethoxy phosphotriesters, 3′-5′ inverted DNA, and chimeric oligonucleotides of mixed backbone composition. Modified bases also pose no problem, as minor bases such as 2′-deoxy-Uridine, 2′-deoxy-Inosine, etheno-containing bases, for example, can be used. Fluorescein and related dyes, spacers, linkers including amino and thiol, sequences with phosphorylation, and other common modifiers have also been used with this invention. Other structures that might be used as well will no doubt be obvious to the skilled artisan and are expected to be covered within the scope of this invention.

In addition to the concentration/desalting protocol, exchange of the cation associated with the nucleic acid can be easily effected using this technique as well. After the salt from the solution has been washed away, a second salt solution containing a new cation can be eluted through the column. The new cation displaces the original cation in a process similar to cation exchange, with the advantage that the procedure takes place on the same column as the concentration/desalting occurred. In a typical process an oligonucleotide purified by anion exchange in which the cation was sodium can be exchanged for ammonium, and indeed, the converse is as straightforward Conventional cation exchange would require a different column with a different solid support that could only be used for cation exchange. Such columns require a recharging of the associated cation in between uses, unlike the methods of the present invention.

Cation exchange can also be accomplished on a nucleic acid which has been lyophilized by dissolving the nucleic acid in an aqueous salt solution, loading onto the column, washing with unbuffered water to remove the unneeded salt, and then washing with a new salt solution containing the new cation. This has the effect of turning the column containing nucleic acid into a cation ion exchange in which the absorbing groups are on the nucleic acid. Conventional cation exchange requires a different column with no other utility, making the use of such a method more time consuming and less cost-effective.

Although applicable to both small volume and large volume samples, the methods of the invention are particularly well suited for large scale concentration and desalting of nucleic acid samples. This is in contrast to other existing techniques, such as precipitation, which are not easily increased in scale. Regardless of whether they are used for small-scale or large-scale production, however, the methods of the invention are rapid, highly reproducible, and give a high level of recovery compared to other methods such as dialysis and diafiltration.

The method of the present invention avoids the use of volatile buffers, significantly reducing the time necessary to complete the procedure as compared to existing methods of reverse phase capture. The present invention also avoids the required use of a separate step involving cation exchange chromatography, precipitation, or other technique to introduce any desired non-volatile cation as a counterion for the nucleic acid. Accordingly, the purified nucleic acid can be obtained directly from the anion exchange pool.

Nucleic Acid Samples

The sample to be purified may be any sample containing the desired nucleic acid, including naturally occurring biological samples and samples from synthesis. In particular, the crude material coming from the synthesis of oligonucleotides after release from the solid phase matrix will, in addition to the desired oligonucleotides and reagents added for the release, also contain water-soluble forms of failure oligonucleotides (i.e., short-mers) formed in unwanted or incomplete reactions during the synthesis. Any method by which these failure sequences can be removed from the sample may be utilized prior to the method of the present invention.

Nucleic acids can be synthesized on commercially purchased DNA synthesizers from <1 uM to >1 mM scales using standard chemistry and methods that are well known in the art, such as Fasman, Practical Handbook of Biochemistry and Molecular Biology, 1989; CRC Press, Boca Raton, Fla., herein incorporated by reference. The practice of the present invention will employ, unless otherwise indicated, conventional techniques of synthetic organic chemistry, biochemistry, molecular biology, and the like, which are within the skill of the art. Such techniques are explained fully in the literature. See, e.g., Sambrook, Fritsch & Maniatis, Molecular Cloning: A Laboratory Manual, Second Edition (1989); Oligonucleotide Synthesis (M. J. Gait, ed., 1984); Nucleic Acid Hybridization (B. D. Hames & S. J. Higgins, eds., 1984); Ansorge et al. (eds) (1997) DNA Sequencing Strategies: Automated and Advanced Approaches (Wiley, NY); and the series, Methods in Enzymology (Academic Press, Inc.).

The described nucleic acids may be partially or filly substituted with any of a broad variety of chemical groups or linkages including, but not limited to: phosphoramidates; phosphorothioates; alkyl phosphonates; 2′-O-methyls; morpholino groups; propyne groups; phosphonates; phosphate esters; phosphoroamidates; 2′-modified RNAs; 3′-modified RNAS; peptide nucleic acids; propynes or analogues thereof or any combination of the above groups or other linkages (or analogues thereof). Synthesis of modified nucleic acids such as phosphoramidite oligonucleotides are disclosed in Stec et al., J. Am Chem. Soc. 106:6077-6089 (1984), Stec et al., J. Org. Chem. 50(20):3908-3913 (1985), Stec et al., J. Chromatog. 326: 263-280 (1985), and LaPlanche et al., Nuc. Acid Res. 14(22):9081-9093 (1986).

The nucleic acids may be completely or partially derivatized by a chemical moiety including, but not limited to, phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate linkages, sulfone internucleotide linkages, 3′-3′ linkages, 5′-2′ linkages, 5′-5′ linkages, 2′-deoxy-erythropentofuranosyl, 2′-fluoro, 2′-O-alkyl nucleotides, 2′-O-alkyl-n(O-alkyl)phosphodiesters, morpholino linkages, p-ethoxy oligonucleotides, PNA linkages, p-isopropyl oligonucleotides, or phosphoroamidates.

Removal of Failure Sequences

A variety of standard methods can be used for the initial purification of the presently described nucleic acids to remove failure sequences, including methods such as those illustrated in U.S. Pat. Nos. 4,430,496, 4,997,927 and 5,395,928, which are incorporated herein by reference. For example, the nucleic acids of the present invention can be purified by chromatography on commercially available reverse phase (for example, see the RAININ Instrument Co., Inc. instruction manual for the DYNAMAX®-300A, Pure-DNA reverse-phase columns, 1989, or current updates thereof, herein incorporated by reference) or ion exchange media such as Waters' Protein Pak or Pharmacia's Source Q (see generally, Warren and Vella, 1994, “Analysis and Purification of Synthetic Nucleic Acids by High-Performance Liquid Chromatography”, in Methods in Molecular Biolog, vol. 26; Protocols for Nucleic Acid Conjugates, S. Agrawal, Ed., Humana Press, Inc., Totowa, N.J.; Aharon et al., 1993, J. Chrom. 698:293-301; and Millipore Technical Bulletin, 1992, Antisense DNA: Synthesis, Purification, and Analysis). Peak fractions can be combined.

A nucleic acid is considered pure when it has been isolated so as to be substantially free of incomplete nucleic acid products produced during the synthesis of the desired nucleic acid. Preferably, a purified nucleic acid will also be substantially free of contaminants which may hinder or otherwise mask the activity of the nucleic acid. In general, where a nucleic acid is able to bind to, or gain entry into a target cell to modulate a physiological activity of interest, it shall be deemed as substantially free of contaminants that would render the nucleic acid less useful.

Protected Oligonucleotides

In one embodiment, the nucleic acid to be purified contains a hydrophobic protecting group. In the purification of such oligonucleotides, it is preferable to have conditions allowing the non-ionic binding between the protected oligonucleotide and the binding material. This means that at low ion concentration both the protected and unprotected oligonucleotides may be adsorbed in this step, although clear advantages are seen in arranging for a selective adsorption of protected oligonucleotides (i.e., a higher salt concentration). Conditions in such techniques are not critical and crude samples may be applied without any prepurification steps. After adsorption, it is preferred to apply a washing step in order to remove non-adsorbed sample constituents including, but not limited to, excess agents from cleavage of the oligonucleotide from the support used during the synthesis. In case both protected and unprotected oligonucleotides have been adsorbed it is advantageous to apply conditions permitting selective desorption of oligonucleotides not carrying the hydrophobic protecting group, e.g., to increase the salt concentration.

Deprotection preferably takes place while the protected oligonucleotide is in an adsorbed state. The conditions are the same as normally applied for each respective protecting group, although it is preferred to keep the conditions so that the formed deprotected oligonucleotides will remain adsorbed (via anion exchange). This normally means that in case the protecting group is transformed to a hydrophobic compound this latter also will remain adsorbed. Typically, the adsorbent is incubated with a cleavage solution matching the protecting group in order for the deprotection to take place. For hydrolytically releasable groups, e.g., DMTr, the solution often contains a relatively strong organic carboxylic acid, such as trifluoroacetic acid, as the cleavage agent. Potentially also dichloro and trichloro acetic acid may be used. In order to secure that the oligonucleotides remain adsorbed, the ionic concentration is normally held as low as possible (often below 0.5M). Typically the temperature and incubation times are between 0 and 40° C. and 1-60 minutes, respectively, bearing in mind that a lower temperature requires a longer incubation time.

Elution of oligonucleotides from hydrophilic anion exchangers is performed using an aqueous solution. The solutions are most preferably water containing appropriate salts (usually inorganic water-soluble salts, such as NaCl) and buffering components. Most preferably the elution is carried out with a salt gradient in order to elute the oligonucleotides according to length. The start and end concentrations as well as the steepness of the gradient will depend on the amount and length of the oligomers to be separated. Elution may also be performed by stepwise changing the ionic strength. Normally, the ionic strength is within in the interval 0-3M and the steepness within the interval 5-40 column volumes.

Protonated/Acidifed Nucleic Acids

Subsequent to, or during, the above synthesis and purification steps, protonated/acidified forms of the described nucleic acids can be generated by subjecting the purified, or partially purified, or crude nucleic acids, to a low pH, or acidic, environment. Purified or crude nucleic acids can be protonated/acidified with acid, including, but not limited to, phosphoric acid, nitric acid, hydrochloric acid, acetic acid, etc. For example, acid may be combined with nucleic acids in solution, or alternatively, the nucleic acids may be dissolved in an acidic solution. Excess acid (may be removed by chromatography or in some cases by drying the nucleic acid.

Desalting and Concentration

The binding material of the method of the invention is a strongly hydrophobic base matrix, such as polydivinylbenzene, poly(styrene-divinylbenzene), polystyrene copolymers, polyethylene, polypropylene, etc., with poly(styrene-divinylbenzene) being the binding material of the preferred embodiment. The use of hydrophobic binding materials which bind strongly to nucleic acids (e.g., oligonucleotides) is crucial to the methods of the invention. Other reverse-phase solid phases (such as C4 and C18) and hydrophobic interaction chromatography phases do not absorb the nucleic acid sufficiently well to allow the use of unbuffered water to wash away the salt to the desired low level.

The binding material is normally porous and may be in particle forms (such as beads) or continuous (monolithic). The particle forms may be used in the form of packed or fluidized beds (expanded beds). In a preferred embodiment, the adsorbent is present as packed beds in a chromatographic column, and even more preferably as fluidized beds in a liquid chromatographic column. Ikuta, et al., Analytical Chemistry 56:2253-2256 (1984); German et al, Analytical Biochemistry 165: 399-405 (1987). For example, any commercially available Hamilton PRP-1 organic reverse phase column may be used in the methods of the invention. This includes PRP-1 columns designed for high pressure liquid chromatography (e.g., columns with 10-20 micron particles) and columns designed for lower pressure liquid chromatography (e.g., columns with 25-75 micron particles). In a preferred embodiment, columns with binding particles in the range of 50-75 microns are used because low pressure columns using this particle size have a high flow rate at a low back-pressure.

Following binding of the nucleic acid to the adsorbent material, the column may be rinsed with an unbuffered aqueous solution to remove the excess salt from the column. Any unbuffered aqueous solution may be used, and preferably the rinsing is performed with neat unbuffered water having 18 Mohm resistance, which is approximately 0 microSiemens/cm conductivity. The column may be rinsed multiple times until the desired effluent conductivity is achieved. It is desirable to achieve a rinse effluent conductivity of at or below 100 microSiemens/cm, since any level above this generally indicates significant amounts of salts remain on the column with the nucleic acid. This salt will elute with the nucleic acid if not removed, and may adversely affect the solution pH and ionic strength of the nucleic acid when resuspended for use, as well as impacting on the secondary structure of the molecule. Thus, it is desirable to achieve a rinse effluent solution of at least below 100 microSiemens/cm, more preferably at least below 50 microSiemens/cm, even more preferably at least below 25 microSiemens/cm.

A number of aqueous organic solvents may be used to elute the nucleic acid in the methods of the invention, including but not limited to acetonitrile, n-propanol, isopropanol, ethanol, or methanol. In a preferred embodiment, aqueous ethanol is the preferred solvent for the method of the invention, since ethanol has a number of advantages: (1) it is environmentally benign; (2) it poses less of a toxicity hazard, and thus is safer to use, than other organic solvents such as acetonitrile; (3) it can be obtained as 95% (190 proof) USP grade for pharmaceutical applications; and (4) it can also preclude the use of antibacterial agents in the desalting process. In a preferred method, the elution solution is 90% aqueous ethanol without any buffering agents. Aqueous alcohol is preferred because mixing undiluted ethanol and water may result in a generation of heat and degassing, which may disrupt a column. While ethanol has several advantages, however, other organic solvents and aqueous solutions of such solvents may be used to elute the nucleic acid in the method of the invention, provided that 1) the solvent allows the nucleic acid to be released from the adsorbent and 2) the nucleic acid is soluble in the solvent.

Once the nucleic acid is desalted and eluted, it can then have the aqueous organic solvent removed, either partially or completely. In general, the elution of the nucleic acid is followed by lyophilization or solvent evaporation under vacuum in commercially available instrumentation such as Savant's Speed Vac. Optionally, small amounts of the nucleic acids may be electrophoretically purified using polyacrylamide gels. Lyophilized or dried-down preparations of nucleic acids can be dissolved in pyrogen-free, sterile, physiological saline (i.e., 0.85% saline), sterile Sigma water, and filtered through a 0.45 micron Gelman filter (or a sterile 0.2 micron pyrogen-free filter).

### EXAMPLES

The present invention and its particular embodiments are illustrated in the following examples. The examples are not intended to limit the scope of this invention but are presented to illustrate and support the claims of this present invention.

### Example 1

### One-Step Concentration and Desalting of a Phosphorothioate Oligonucleotide

A phosphorothioate 21-mer oligonucleotide was previously purified by strong anion exchange chromatography. The solvents used were based on aqueous sodium chloride, with a pH of 12 to disrupt any secondary structure. A small amount (5%) of ethanol had been added to the elution buffer to assist with the elution. Fractions of the SAX eluent were pooled to prepare an oligonucleotide containing solution that was approximately 1M sodium chloride, 2% ethanol, at a pH of 12, with a concentration of oligonucleotide of 11 A₂₆₀/ml, total volume of 3 L, or approximately 35,000 A260 which is approximately 1 g of oligonucleotide phosphorothioate. The ethanol was removed by partial drying and the volume reduced by about 10%, resulting in an increase of concentration of oligonucleotide to 12.8 A₂₆₀/ml. The oligonucleotide solution was loaded onto a low-pressure column of Hamilton 50-75 micron PRP-1 in an Amicon Vantage column of 4.4×30 cm at a flow rate of 60 ml/min. Loading was complete in less than 1 hour, at which time 100 ml of 0.6M sodium chloride, pH 12, was used to rinse the loading system. The solvent was changed to unbuffered water (18.2 Mohm) and the column washed at 24 ml/min until the conductivity was 25 microSiemens/cm, a drop from the 80 microSiemens/cm observed during the loading. At this time a gradient of 0-70% B (B=90% ethanol, denatured) in 14 minutes was started at the same flow rate. Fractions were collected of the eluent while monitoring the absorbance at 254 nm. After elution the fractions were combined and assayed for yield. The fractions containing oligonucleotide had a volume of 300 ml after combining, with 33,500 A₂₆₀ recovered.

### Example 2

### One-Step Concentration and Desalting of a DNA Oligonucleotide

A phosphodiester 20-mer oligonucleotide was previously purified by strong anion exchange chromatography. The solvents used were based on aqueous sodium chloride, with a pH of 12 to disrupt any secondary structure. Fractions of the SAX eluent were pooled to prepare an oligonucleotide-containing solution that was approximately 1M sodium chloride, at a pH of 12, with a concentration of oligonucleotide of 1.4 A₂₆₀/ml, total volume of 118 ml, for a total of 170 A₂₆₀ which is approximately 6 mg of oligonucleotide phosphodiester. The oligonucleotide solution was loaded onto a low-pressure column of Hamilton 50-75 micron PRP-1 in an Amicon Vantage column of 1.6×30 cm at a flow rate of 12 ml/min. When loading was complete 5 ml of 0.3M sodium chloride, pH 12, was used to rinse the loading system. The solvent was changed to unbuffered water (18.2 Mohm) and the column washed at 3 ml/min until the conductivity was 25 microSiemens/cm. At this time a gradient of 0-70% B (B=90% ethanol, denatured) in 14 minutes was started at the same flow rate. The absorbance of the eluent at 254 nm was monitored, and the eluent containing oligonucleotide collected in a single portion. The recovered oligonucleotide (153 A₂₆₀) was then lyophilized.

### Example 3

### Acidification of a 2′-O-methyl RNA Oligonucleotide

A 21-mer 2′-O-methyl RNA was previously purified by strong anion exchange chromatography. The solvents used were based on aqueous sodium chloride, with a pH of 12 to disrupt any secondary structure. Fractions of the SAX eluent were pooled to prepare an oligonucleotide-containing solution that was approximately 1M sodium chloride, at a pH of 12, with a concentration of oligonucleotide of 11 A₂₆₀/ml, total volume of 70 ml, or approximately 750 A₂₆₀ which is approximately 25 mg of oligonucleotide. The oligonucleotide solution was loaded onto a medium-pressure column of Polymer Labs PLRP in a Waters AP-1 column of 1×30 cm at a flow rate of 12 ml/min. After loading was complete, 12 ml of 0.6M sodium chloride, pH 12, was used to rinse the loading system. When the rinsing was complete, the oligonucleotide was washed first with 18 ml of aqueous 0.4M NaCl-25 mM HCl, followed by 18 ml aqueous 25 mM HCl. The solvent was changed to unbuffered water (18.2 Mohm) and the column washed at 1.5 ml/min until the conductivity was 10 microSiemens/cm. At this time a gradient of 0-40% B (B=90% ethanol, denatured) in 20 minutes was started at the same flow rate. The absorbance of the eluent at 254 nm was monitored, and the eluent containing oligonucleotide collected in a single portion. The recovered oligonucleotide (684 A₂₆₀) now had a pH of 2.5-3 when dissolved in water at a concentration of 30 A₂₆₀/ml (app. 1 mg/ml).

### Example 4

### Exchange of Ammonium for Sodium Counterion of a Phosphorothioate Oligonucleotide

A 21-mer phosphorothioate oligonucleotide was previously purified by strong anion exchange chromatography under conditions in which the counterion was sodium. The oligonucleotide (979 A₂₆₀) was dissolved in 36 ml of 0.6M NaCl, pH 12. The oligonucleotide solution was loaded onto a low-pressure column of Hamilton 50-75 micron PRP-1 in an Amicon Vantage column of 1.6×30 cm at a flow rate of 12 ml/min. After loading was complete, 10 ml of 0.6M sodium chloride, pH 12, was used to rinse the loading system. When the rinsing was complete, the flow rate was dropped to 3 ml/min and the column washed with unbuffered water (18.2 Mohm) until the conductivity was 25 microSiemens/cm. At this time 60 ml (1 column volume) of 2M NH₄Cl washed through the column at 3 ml/min, followed by additional water. When the conductivity dropped to 16 microSiemens/cm after the NH₄Cl washed through the column, a gradient of 0-70% B (B=90% ethanol, denatured) in 14 minutes was staed at the same flow rate. The absorbance of the eluent at 254 nm was monitored, and the eluent containing oligonucleotide collected in a single portion using a fraction collector. The recovered oligonucleotide (851 A₂₆₀ in 33 ml) as the ammonium salt was then ready for lyophilization.

In these examples the amounts of oligonucleotide are indicated in units. While these units are extensively used in the field as units of measure for oligonucleotides, the extinction coefficients on which these measurements are based are sensitive to pH, solvent effects, oligonucleotide molecular interactions, and amounts of salts present in the sample. As such, the use of units are intended for illustration purposes in the above examples rather than as absolute values.

Although the present invention has been described with reference to specific examples, they are in no way to be construed as limiting the reagents and processes of the present invention. It will be appreciated by persons skilled in the art that the present invention is not limited to what has been shown and described herein above, but it is to be determined solely in terms of the following claims.

1. A method of desalting and concentrating a nucleic acid within a sample, said method comprising the steps of:
contacting the sample with a binding medium comprising a strongly hydrophobic base matrix; and
eluting the nucleic acid with an aqueous organic solvent.

2. The method of claim 1, wherein the binding medium is comprised of poly(styrene-divinylbenzene).

3. The method of claim 1, wherein the binding medium is a column comprised of particles having a diameter of about 1 micron to about 250 microns.

4. The method of claim 3, wherein the binding medium is a column comprised of particles having a diameter of about 50 to about 75 microns.

5. The method of claim 1, further comprising the step of:
rinsing the binding medium with an unbuffered aqueous solution prior to elution.

6. The method of claim 5, wherein the unbuffered aqueous solution is water.

7. The method of claim 5, wherein an effluent conductivity following rinsing is at or below 100 microSiemens/cm.

8. The method of claim 7, wherein the effluent conductivity following rinsing is at or below 25 microSiemens/cm.

9. The method of claim 1, wherein the nucleic acid has been modified with a compound selected from the group consisting of: biotin, fluorescein and related dyes, spacers, thiol modifiers, amino modifiers, carboxylate modifiers, or any combination of these.

10. The method of claim 1, wherein the nucleic acid is selected from the group consisting of: a DNA phosphodiester, RNA phosphodiester, phosphorothioate, methylphosphonate, 2′-methyl RNA, 2′-O-alkyl RNA, 2′-O-methyl DNA, 2′-O-alkyl DNA and chimeras containing such structures.

11. The method of claim 1, wherein the nucleic acid comprises nucleotide bases selected from the group consisting of: 5-methylcytidine, inosine, halogenated uridines, etheno-bases, dideoxynucleosides, and inverted bases.

12. The method of claim 1, wherein the nucleic acid is comprised of inverted 3′-5′ linkages.

13. The method of claim 1, wherein the nucleic acid is comprised of 5′-2′ linkages.

14. The method of claim 1, wherein the nucleic acid is an oligonucleotide comprised of about 1 to about 100 nucleotides.

15. The method of claim 1, wherein the sample is the product of strong anion exchange chromatography.

16. The method of claim 1, wherein the sample is the product of weak anion exchange chromatography.

17. The method of claim 1, wherein the sample is derived from a biological source material.

18. The method of claim 1, wherein the aqueous organic solvent is selected from the group consisting of acetonitrile, n-propanol, isopropanol, or methanol.

19. The method of claim 1 wherein the aqueous organic solvent is aqueous ethanol.

20. A method of exchanging a cation associated with a nucleic acid in a sample, comprising the steps of:
contacting a nucleic acid associated with a first cation with a binding medium comprising a strongly hydrophobic base matrix;
rinsing the nucleic acid bound to the binding medium with an unbuffered aqueous solution prior to elution;
contacting the bound nucleic acid with a solution comprised of a second cation; and
eluting the nucleic acid associated with the second cation from the binding medium;
wherein the second cation effectively displaces the first cation in the effluent sample.
